(19) United States
(12) Patent Application Publication
(10) Pub. No.: US 2017/0164890 A1
(43) Pub. Date: Jun. 15, 2017

(54) SYSTEM TO FACILITATE THERAPEUTIC POSITIONING FOR A BODY PART

(71) Applicant: Intel Corporation, Santa Clara, CA (US)

(72) Inventors: TERRENCE P. LEIP, Chandler, AZ (US); CHRISTOPHER J. MCCONNELL, Gilbert, AZ (US)

(73) Assignee: Intel Corporation, Santa Clara, CA (US)

(21) Appl. No.: 14/966,197

(22) Filed: Dec. 11, 2015

(51) Int. Cl. A61B 5/00; G06F 3/16; A61B 5/11; G02C 11/00; G09B 5/02; G06F 3/01; G06F 3/0481

(52) U.S. Cl. CPC A61B 5/486; G06F 3/011; G06F 3/16; G06F 3/0481; G02C 11/10; G09B 5/02; A61B 5/6803; A61B 5/1114; A61B 5/742; A61B 5/7405; A61B 2560/0223

(57) ABSTRACT

The present disclosure pertains to a system to facilitate therapeutic positioning for a body part. An example system may be wearable by a user (e.g., a patient) and may comprise at least a sensor and presentation circuitry. The sensor may sense a position corresponding to a body part of the patient and generate an electronic signal based on the position. The presentation circuitry may then present a notification (e.g., display visible indicia and/or generate sound) to instruct the patient how to move the body part into a therapeutic position. For example, control circuitry in the system may receive the electronic signal from the sensor, compare the determined position to the therapeutic position, generate the notification and provide the notification to the presentation circuitry.

### TECHNICAL FIELD

The present disclosure relates to electronic positioning, and more particularly, to a system for assisting a patient to maintain a prescribed position of a body part to facilitate proper healing.

### BACKGROUND

Even if a medical procedure (e.g., surgery) is performed to remedy a medical condition, a period of convalescence may be prescribed following the procedure. This period of time may be required to allow a patient to fully heal. In some instances a medical practitioner (e.g., a doctor, a nurse, a physical therapist, etc.) may instruct the patient to maintain a body part in a particular position, orientation, etc. This position, orientation, etc. may be relative (e.g., with respect to the rest of the patient's body) or absolute (e.g., at a certain position or angle with respect to a fixed coordinate system). At least one procedure where this may be essential is reattaching a detached retina. After the doctor suture's the detached retina back into place, air may be injected into the eye of the patient to provide gentle pressure allowing the retina to heal in the correct position. To facilitate correct positioning of the air bubble within the eye, the doctor may specify that the patient maintain his/her head in a certain position for certain durations of time during each day. Other examples may include a patient maintaining a broken limb, repaired joint (e.g., a repaired anterior cruciate ligament (ACL) in a knee joint), etc. in a certain position to encourage healing, reduce swelling, etc.

While following a prescribed positioning routine for a body part may sound simple, the actual execution may be difficult. The body part of the patient may need to be maintained in the prescribed position, orientation, etc. for long amounts of time. The patient may lose awareness of this requirement when performing other activities. Moreover, there is no way for a patient to realize that the body part has left the prescribed position when they are sleeping. A patient may, for example, move their body part in a totally unintentional manner during sleep. This may be prevented in some instances where physical restraints may be used to ensure that the prescribed position in maintained, but in some instances the usage of physical restraints may be impossible (e.g., due to the nature of the procedure or the body part on which the procedure was performed) or potentially damaging where the patient may be experiencing totally unintentional movement.

### BRIEF DESCRIPTION OF THE DRAWINGS

Features and advantages of various embodiments of the claimed subject matter will become apparent as the following Detailed Description proceeds, and upon reference to the Drawings, wherein like numerals designate like parts, and in which:

FIG. 1 illustrates an example system to facilitate therapeutic positioning for a body part in accordance with at least one embodiment of the present disclosure;

FIG. 2 illustrates an example application of the system of FIG. 1 in accordance with at least one embodiment of the present disclosure; and

FIG. 3 illustrates example operations for facilitating therapeutic positioning for a body part in accordance with at least one embodiment of the present disclosure.

Although the following Detailed Description will proceed with reference being made to illustrative embodiments, many alternatives, modifications and variations thereof will be apparent to those skilled in the art.

### DETAILED DESCRIPTION

The present disclosure pertains to a system to facilitate therapeutic positioning for a body part. An example system may be wearable by a user (e.g., a patient) and may comprise at least a sensor and presentation circuitry. The sensor may sense a position corresponding to a body part of the patient and generate an electronic signal based on the position. The presentation circuitry may then present a notification (e.g., display visible indicia and/or generate sound) to instruct the patient how to move the body part into a therapeutic position. For example, control circuitry in the system may receive the electronic signal from the sensor, compare the determined position to the therapeutic position, generate the notification and provide the notification to the presentation circuitry. In at least one embodiment, the sensor, presentation circuitry and control circuitry may be coupled to a structure that is worn by the patient such as, for example, eyeglasses, a headband, etc. The system may further be configured to perform operations including, for example, sensor calibration, updating the therapeutic position based on data received from outside of the system, exporting stored positioning data corresponding to the determined position of the body part, etc.

In at least one embodiment, an example system for therapeutic positioning of a body part may comprise a sensor and presentation circuitry. The sensor may be wearable by a user and may sense a position of a user body part on which the sensor is worn and generate an electronic signal indicative of the position. The presentation circuitry may also be wearable by the user and may present at least one notification instructing how the body part should be moved based on the electronic signal to relocate the body part from the position to a therapeutic position.

In at least one embodiment, at least a portion of the presentation circuitry may be located in a first presentation device positioned in front of an eye of the user, and the notification may cause the first presentation device to present visible indicia instructing how to move the body part. At least a portion of the presentation circuitry may also be located in a second presentation device to at least generate sound, and the notification may cause the second presentation device to generate audible indications instructing how to move the body part. The system may further comprise control circuitry to, for example, at least receive the electronic signal, determine the position of the body part based on the electronic signal, generate the notification based on the position and provide the notification to the presentation circuitry. The system may further comprise communication circuitry coupled to at least the control circuitry to at least one of receive data regarding at least the therapeutic position or transmit data regarding the determined position of the body part. At least the control circuitry and communication circuitry may be located in a data processing device wearable by the user, the control circuitry interacting with at least one of the first or second presentation circuitry via the communication circuitry. The system may further comprise user interface circuitry coupled to at least the control circuitry, wherein the user interface circuitry includes at least the sensor.

In at least one embodiment, at least the sensor, the presentation circuitry and control circuitry may all be coupled to an apparatus worn by the user. For example, the apparatus may be a headband and the body part is the head of the user. Alternatively, the apparatus may be eyeglasses, the body part is the head of the user, the first presentation device is coupled to an eyeglass lens in the eyeglasses and the control circuitry and second presentation device are coupled to a frame of the eyeglasses. Consistent with the present disclosure, an example method for therapeutic positioning of a body part may comprise activating sensing for a position of a user body part, sensing the position, generating an electronic signal corresponding to the position, comparing the position to a therapeutic position and presenting at least one notification to relocate the body part from the position to the therapeutic position based at least on the comparison.

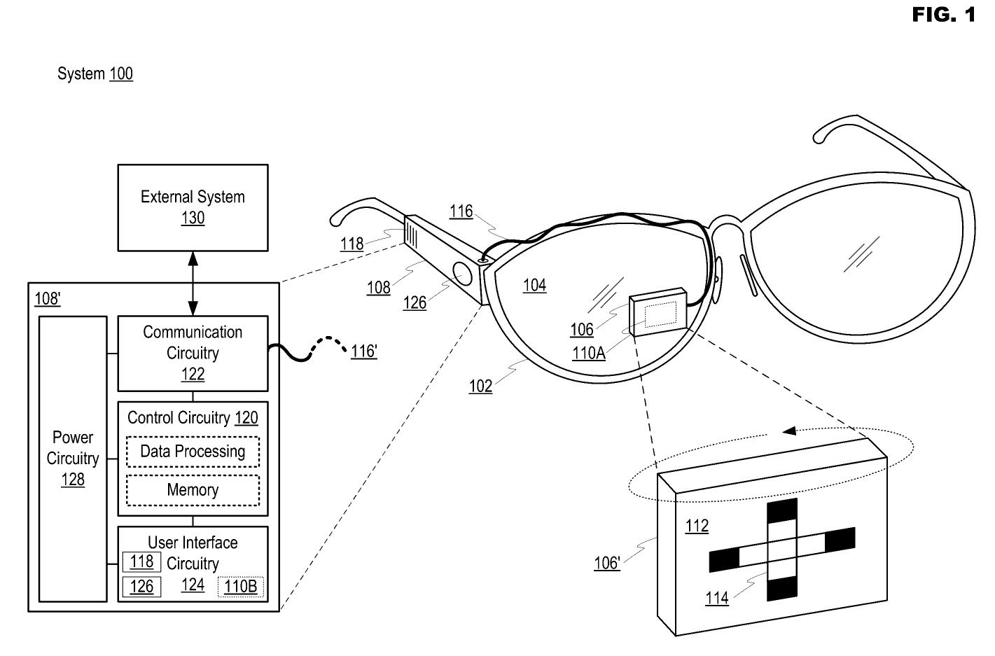

FIG. 1 illustrates an example system to facilitate therapeutic positioning for a body part in accordance with at least one embodiment of the present disclosure. While various example implementations, technologies, etc. may be referenced herein, the references are made merely to provide a readily comprehensible perspective from which the more generalized devices, systems, methods, etc. taught herein may be understood. Other applications, configurations, technologies, etc. may result in different implementations still consistent with the teachings presented herein. As referenced herein, the term “position” may generally refer to both a position of a body part with respect to a fixed or relative coordinate system and/or the orientation of the body part.

FIG. 1 illustrates an example implementation wherein system 100 is implemented using a pair of eyeglasses (e.g., to monitor the head position of a patient that has had retina reattachment surgery). Eyeglasses may make an appropriate foundation on which features consistent with the present disclosure may be implemented. Moreover, since eyeglasses, sunglasses, safety glasses, etc. are already routinely available and worn by people, it also means that there is little barrier to adoption of the technology. Most of the equipment in system 100 may be incorporated within, or at least mounted onto, a pair of eyeglasses, and so the amount of equipment that patients need to actually carry may be minimal. Notwithstanding the foregoing advantages offered by eyeglasses of the types discussed above, the teachings disclosed herein may alternatively be embodied in different form factors depending on, for example, the type of the convalescence being monitored.

The eyeglasses shown in FIG. 1 may comprise frame 102 and lenses 104. In at least one example implementation, a patient having surgery may have system 100 applied to his/her own prescription eyeglasses to aid in post-surgery recuperation. Alternatively, a patient may be given eyeglasses that do not comprise prescription lenses 104, an apparatus that is specifically designed for the condition being monitored (e.g., into which the devices in system 100 may be embedded) such as a wearable structure like a frame, headband, etc. In at least one embodiment system 100 may comprise at least one sensor and presentation circuitry. In the example implementation that is shown in FIG. 1, the presentation circuitry is divided between a first presentation device 106 and a second presentation device 108. While not shown, it may also be possible for the at least one sensor presentation circuitry to reside in one device (e.g., in first presentation device 106).

The at least one sensor (generally, “sensor 110”) is shown in two possible locations 110A and 110B in FIG. 1. Sensor 110 may be configured to generate an electronic signal indicative of position, orientation, impact, motion (e.g., including direction, speed and/or acceleration), etc. A variety of technologies may be employed to implement sensor 110 such as, but not limited to, electronic, magnetic, electromagnetic and/or electromechanical position, orientation, direction, speed, acceleration or impact sensors, absolute position and/or orientation sensors based on a fixed coordinate system (e.g., Global Positioning System (GPS), magnetic compass headings, etc.), relative position and/or orientation determination (e.g., based on electronic signal sensing such as direction of arrival estimation), etc. The implementation of sensor 110 in location 110A or 110B may depend on factors such as the number of devices being implemented in system 100, resource constraints (e.g., power, size, processing, etc.) in each of the devices in system 100, etc. Moreover, it may be possible for sensor 110 to be implemented in a location separate from the other portions of the system 100. For example, sensor 110 may be removably affixed to the part of user's body for which treatment is required (e.g., arm, leg, back, etc.) while the remainder of system 100 is embodied in a manner such as illustrated in FIG. 1. Sensor 110 may communicate with the remainder of system 100 via wired or wireless communication. In such a configuration, for example, a user may be notified via first presentation device 106 and/or second presentation device 108 that an injured extremity is not currently positioned therapeutically (e.g., to promote healing), that the current position of their spine is not conducive to having good posture, etc.

An example implementation of first presentation device 106 is shown at 106′ in FIG. 1 in an orientation rotated 180 degrees so that surface 112, which would ordinarily face towards the patient when system 100 is being worn, is visible. For example, surface 112 may adhere to lens 104 via a removable adhesive, a mechanical coupling (e.g., a clip, strap, rubber band, etc.), etc. so that the patient may be able to view surface 112 through lens 104. First presentation device 106 may comprise at least display 114 visible on surface 112. Display 114 may allow visible indicia instructing how to move a body part to be presented to the patient. Display 114 may include, for example, a series of light emitting diodes (LEDs) indicating the position of a body part of the patient wearing system 100. When the body part of the patient is determined to be in a therapeutic position a central LED may be illuminated (e.g., at least momentarily to confirm proper positioning). As referenced herein, a “therapeutic position” may be a position prescribed for a body part to promote proper healing of the body part or another body part influenced by the position of the body part. The therapeutic position may be prescribed by a medical practitioner, who may instruct the patient to maintain the therapeutic position for a certain period of time to promote proper healing. If the body part is determined to not be in the therapeutic position, at least one periphery LED may be illuminated to indicate the direction in which the body part must be moved to achieve correct position. In FIG. 1, the grey boxes in display 114 may correspond to LEDs indicating a small movement is required, while the black blocks in display 114 may correspond to LEDs indicating that a more substantial movement is required. The illumination of multiple proximate boxes may indicate that, for example, diagonal movement of the body part is required, that the amount/direction of movement required to place the body part back into the therapeutic position falls between the amounts of movement defined by the LEDs, between two of the four directions defined by the LEDs, etc. While an LED array is illustrated in regard to display 114 in FIG. 1, other implementations of display 114 may include, but are not limited, a visible interface including different shaped features that may illuminate (e.g., directional arrows that may illuminate to show direction), displays capable of presenting variable types of visible indicia such as LED matrix displays, active-matrix organic light-emitting diode, (AMOLED) displays, liquid crystal displays (LCDs), plasma displays, electronic paper (e-paper) displays, etc.

Second presentation device 108 is shown coupled to frame 102, and combines at least a portion of the presentation circuitry (e.g., at least speaker 118 and any supporting circuitry) with data processing circuitry. While some of presentation and data processing circuitry are shown as combined in FIG. 1, this is merely for the sake of explanation. Other configurations are possible including, for example, speaker 118 being contained in a separate device (e.g., earpiece) coupled via a wired or wireless link to device 108′ that includes the data processing circuitry. It may also be possible for some or all of the data processing circuitry to be located in external system 130. In such an implementation, the wearable circuitry of system 100 would mainly comprise sensor 110, first presentation device 106′ and second presentation device 108′ along with some limited circuitry to at least support the interaction with external system 130. Most of the data processing would then be handled by external system 130 (e.g., a smart phone, tablet computer, etc.). In at least one embodiment, second presentation device 108 may be configured to at least receive an electronic signal from sensor 110, determine a position based on the electronic signal, compare the position to a therapeutic position and generate a notification based on the comparison. In addition to the visible indicia set forth above, notifications may further comprise audible indications instructing how to move the body part that are generated by speaker 118. Audible indications may include, for example, audible tones that alert the patient, provide an indication as to how the body part should be moved, etc. recorded movement instructions, etc. One or both of the visible or audible indicia may be enhanced with tactile feedback (e.g., vibration) to alert the patient of a deviation in body part position. In at least one embodiment, the patient may be able to configure whether the notification will be visible, audible and/or tactile based on time of day, location (e.g., GPS sensing) and/or current activity. For example, a patient may configure visible only notifications when in a public place, configure audible only notifications when sleeping, etc.

As shown in FIG. 1, second presentation device 108′ may comprise, for example, control circuitry 120, communication circuitry 122, user interface circuitry 124 and power circuitry 128. Control circuitry 120 may include at least data processing and memory resources. For example, data processing resources may include one or more processors situated in separate components, or alternatively one or more processing cores embodied in a component (e.g., in a System-on-a-Chip (SoC) configuration), and any processor-related support circuitry (e.g., bridging interfaces, etc.). Example processors may include, but are not limited to, various x86-based microprocessors available from the Intel Corporation including those in the Pentium®, Xeon®, Itanium®, Celeron®, Atom®, Quark®, Core i-series, product families, Advanced RISC (e.g., Reduced Instruction Set Computing) Machine or “ARM” processors, etc. Examples of support circuitry may include chipsets (e.g., Northbridge, Southbridge, etc. available from the Intel Corporation) to provide an interface through which the data processing resources may interact with other system components that may be operating at different speeds, on different buses, etc. in second presentation device 108. Some or all of the functionality commonly associated with the support circuitry may also be included in the same physical package as the processor (e.g., such as in the Sandy Bridge family of processors available from the Intel Corporation).

In at least one embodiment, the data processing resources may be configured to execute various instructions in second presentation device 108. Instructions may include program code configured to cause the data processing resources to perform activities related to reading data, writing data, processing data, formulating data, converting data, transforming data, etc. Information (e.g., instructions, data, etc.) may be stored in the memory resources. The memory resources may comprise random access memory (RAM) or read-only memory (ROM) in a fixed or removable format. RAM may include volatile memory configured to hold information during the operation of second presentation device 108 such as, for example, static RAM (SRAM) or Dynamic RAM (DRAM). ROM may include non-volatile (NV) memory circuitry configured based on BIOS, UEFI, etc. to provide instructions when second presentation device 108 is activated, programmable memories such as electronic programmable ROMs (EPROMS), Flash, etc. Other fixed/removable memory may include, but are not limited to, magnetic memories such as, for example, floppy disks, hard drives, etc., electronic memories such as solid state flash memory (e.g., embedded multimedia card (eMMC), etc.), removable memory cards or sticks (e.g., micro storage device (uSD), USB, etc.), optical memories such as compact disc-based ROM (CD-ROM), Digital Video Disks (DVD), Blu-Ray Disks, etc.

Communication circuitry 122 may include resources configured to support wired and/or wireless communications. In at least one example implementation, second presentation device 108 may comprise multiple sets of communication circuitry 122 including, for example, separate physical interface circuitry for wired protocols and/or wireless radios. Wired communications may include, for example, serial and parallel wired mediums such as Ethernet, Universal Serial Bus (USB), Firewire, Thunderbolt, Digital Video Interface (DVI), High-Definition Multimedia Interface (HDMI), etc. Wireless communications may include, for example, close-proximity wireless mediums (e.g., radio frequency (RF) such as based on the RF Identification (RFID) or Near Field Communications (NFC) standards, infrared (IR), etc.), short-range wireless mediums (e.g., Bluetooth, WLAN, Wi-Fi, etc.), long range wireless mediums (e.g., cellular wide-area radio communications, satellite-based communications, etc.), electronic communications via sound waves, etc. In one embodiment, communication circuitry 122 may be configured to prevent wireless communications from interfering with each other. In performing this function, communication circuitry 122 may schedule communication activities based on, for example, the relative priority of messages awaiting transmission.

User interface circuitry 124 may include hardware and/or software to allow users to interact with second presentation device 108 such as, for example, various input mechanisms (e.g., microphones, switches, buttons, knobs, keyboards, speakers, touch-sensitive surfaces, one or more sensors configured to capture images and/or sense proximity, distance, motion, gestures, orientation, biometric data, etc.) and various output mechanisms (e.g., speakers, displays, lighted/flashing indicators, electromechanical components for vibration, motion, etc.). The hardware in user interface circuitry 124 may be incorporated within second presentation device 108 and/or may be coupled to second presentation device 108 via a wired or wireless communication medium. Power circuitry 128 may include internal power sources (e.g., battery, fuel cell, etc.) and/or external power sources (e.g., power grid, electromechanical or solar generator, external fuel cell, etc.) and related circuitry configured to supply second presentation device 108 with the power needed to operate. In FIG. 1, user interface circuitry may comprise, for example, at least speaker 118, control button 126 (e.g., to activate system 100, to control the operation of system 100, etc.) and possibly sensor 110B, depending on the device configuration.

External system 130 may include equipment that is at least able to provide the therapeutic position to second presentation device 108′ via communication circuitry 122 and/or to receive sensed position data from second presentation device 108′ via communication circuitry 122. Examples of external system 130 may include, but are not limited to, a mobile communication device such as a cellular handset or a smartphone based on the Android® OS from the Google Corporation, iOS® or Mac OS® from the Apple Corporation, Windows® OS from the Microsoft Corporation, Linux® OS, Tizen® OS and/or other similar operating systems that may be deemed derivatives of Linux® OS from the Linux Foundation, Firefox® OS from the Mozilla Project, Blackberry® OS from the Blackberry Corporation, Palm® OS from the Hewlett-Packard Corporation, Symbian® OS from the Symbian Foundation, etc., a mobile computing device such as a tablet computer like an iPad® from the Apple Corporation, Surface® from the Microsoft Corporation, Galaxy Tab® from the Samsung Corporation, Kindle® from the Amazon Corporation, etc., an Ultrabook® including a low-power chipset from the Intel Corporation, a netbook, a notebook, a laptop, a palmtop, etc., a wearable device such as a wristwatch form factor computing device like the Galaxy Gear® from Samsung, Apple Watch® from the Apple Corporation, etc., a typically stationary computing device such as a desktop computer, a server, a group of computing devices organized in a high performance computing (HPC) architecture, a smart television or other type of “smart” device, small form factor computing solutions (e.g., for space-limited applications, TV set-top boxes, etc.) like the Next Unit of Computing (NUC) platform from the Intel Corporation, etc. or combinations thereof.

While FIG. 1 shows an example implementation wherein first presentation device 106′ is linked to second presentation device 108′ via wire 116, the link may also be a wireless (e.g., via Bluetooth). In an example of operation, first presentation device 106 may be coupled to lens 104 and second presentation device 108′ may be coupled to frame 102 (e.g., of a pair of the patient's eyeglasses, of nonprescription glasses, etc.). System 100 may then be activated by, for example, pressing control button 126. In at least one embodiment, at least the initial activation of system 100 may be followed by configuration and/or calibration operations. These operations may only be necessary for some systems 100. For example, a fixed system 100 (e.g., wherein the sensor, presentation circuitry, data processing circuitry, etc. are permanently incorporated in a wearable structure that may be dedicated to tracking body part position) may not require one or either of the configuration and/or calibration operations since the location of various circuitry making up system 100 does not change. Configuration operations may comprise configuring at least one configuration setting in system 100 including, for example, whether first presentation device 106′ and second presentation device 108′ are present, active, etc. in system 100, the location of first presentation device 106′ in regard to the eyeglasses and/or body part of the patient, setting up communications between second presentation device 108′ and first presentation device 106′ and/or external system 130, loading a therapeutic position from external system 130, configuring a schedule for the patient to maintain the therapeutic position, etc. The configuration operations may be performed using, for example, an external user interface (e.g., a user interface within, or coupled to, external system 130). The configuration operations may be followed by calibration operations that may calibrate the operation of sensor 110 and/or the data processing circuitry based on the position of sensor 110 (e.g., at location 110A or 110B). After configuration and/or calibration operations are complete (e.g., if necessary), system 100 may enter normal operation wherein sensor 110 may generate an electronic signal corresponding to the body part on which sensor 110 is worn. The electronic signal may be provided to the data processing circuitry in second presentation device 108′, which may proceed to determine a position for the body part based on the electronic signal, compare the determined position to the therapeutic position and generate a notification based on the comparison. The notification may then be provided to first presentation device 106′, speaker 118 and/or a electromechanical device in system 100 so that visible, audible and/or tactile feedback may be provided to the patient to correct the position of the body part to correspond to the therapeutic position.

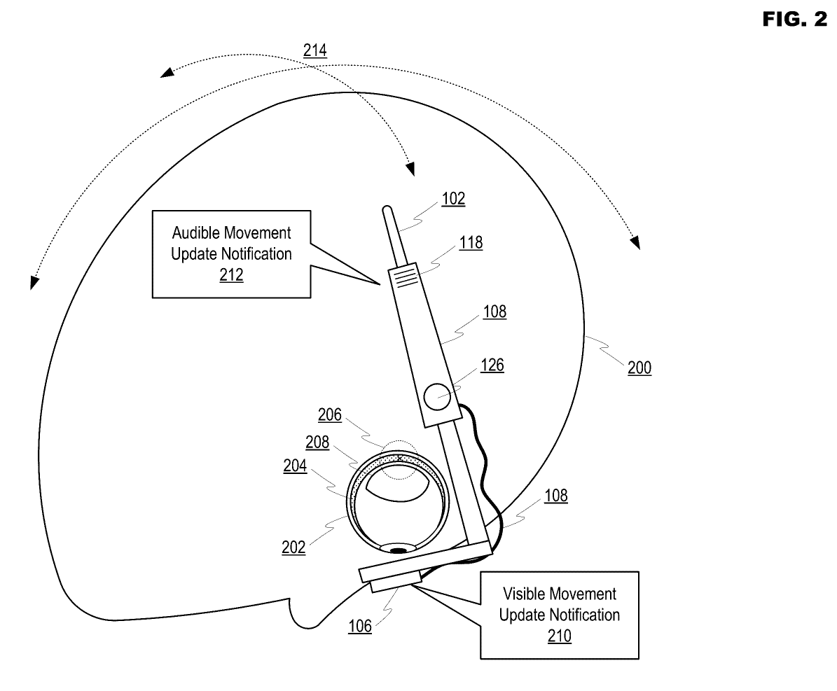

FIG. 2 illustrates an example application of the system of FIG. 1 in accordance with at least one embodiment of the present disclosure. While the various embodiments disclosed herein may be applied to the positioning of any body part to help promote proper healing, an application wherein a patient has sustaining a detached retina provides a readily comprehensible scenario for use in explaining an example embodiment. Various elements of system 100 that were described in regard to FIG. 1 are repeated in FIG. 2 for reference, and maintain the same reference numbers as in FIG. 1. Head 200 of a patient is shown in FIG. 2 comprising eye 202. Eye 202 may include retina 204 which has become detached. During the procedure to reattach the retina, a doctor may position the detached portion of retina 204 (e.g., as shown at 206) by injecting gas into the eye to create bubble 208. Bubble 208 may hold the torn portion of retina 204 in place while the doctor repairs the retina (e.g., seals the tear) using cryopexy (e.g., a freezing probe) or photocoagulation (e.g., a laser beam). As part of recuperation, the patient needs to maintain head 200 in a position that maintains bubble 208 under the repaired portion 206 of retina 204. The buoyancy of bubble 208 provides upward pressure on the repaired portion 206 of retina 204, which may help promote healing. The location of bubble 208 in eye 202 depends on the position of head 200. Consistent with the present disclosure, at least one of visible movement update notifications 210 or audible movement update notifications 212 may be generated by system 100 (e.g., with or without tactile feedback) to cause the patient to change position 214 of head 200 to a therapeutic position (e.g., so that bubble 208 is maintained in contact with at least repaired portion 206 of retina 204). As mentioned above, the operation of system 100 may be configured to generate notifications for certain periods of time to promote healing without putting strain on the patient. Moreover, in at least one embodiment system 100 may store data indicating at least one determined position 214 of head 200, and may provide the information to external system 130. The determined position data may allow medical practitioner to contact a patient or request that the patient come in for an examination if, for example, a duration of time the body part has spent in the therapeutic position is deemed inadequate for proper healing, if a position, impact, movement, etc. was detected that may adversely affect healing, etc. The medical practitioner may also be able to update or change the therapeutic position to account for changes in the patents situation. The updated therapeutic position may, for example, be provided to external system 130 (e.g., to a patient's smart phone or other computing device via a wide-area network (WAN) like the Internet, a local area network (LAN), etc.) by the medical practitioner. External device 130 may then be used to update the therapeutic position that is stored on second presentation device 108.

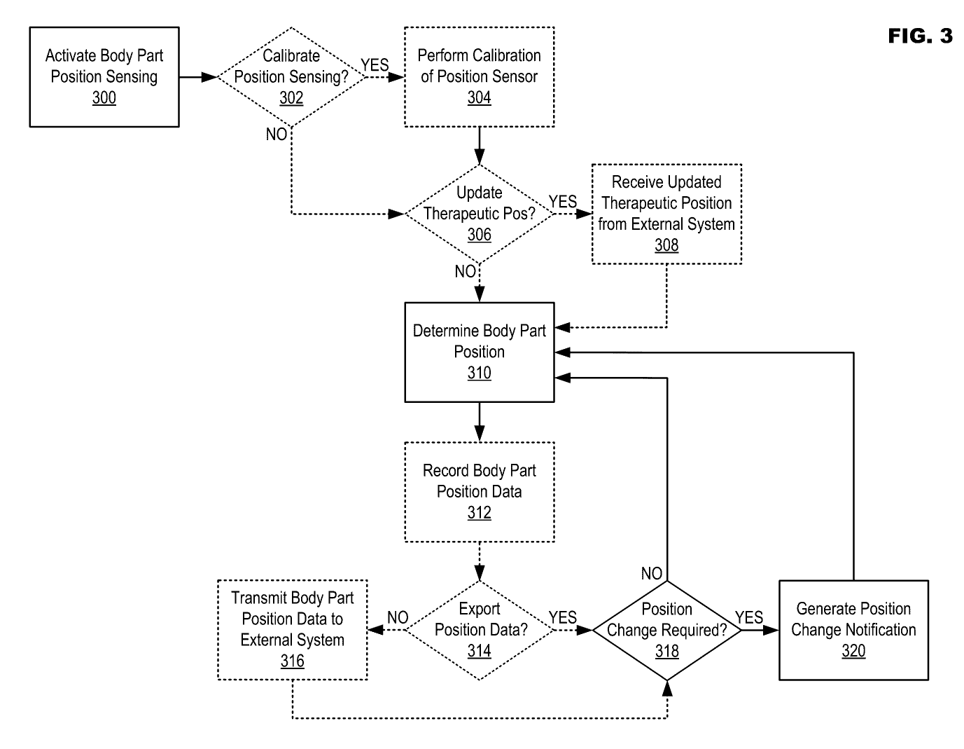

FIG. 3 illustrates example operations for facilitating therapeutic positioning for a body part in accordance with at least one embodiment of the present disclosure. Operations shown in FIG. 3 with dotted lines may be optional in that implementation of these operations may depend on, for example, intended usage for the system (e.g., the body part, malady, treatment, etc.), the configuration of the system, the abilities of each of the devices incorporated in the system, etc. In operation 300 body part position sensing may be activated. Operations 302 to 304 pertain to calibrating position sensing. Position sensing calibration may be applicable to certain situations such as, for example, where the system may be coupled to an existing wearable structure like a patient's prescription eyeglasses, nonprescription eyewear, a bone setting apparatus (e.g., a cast, brace, external bone fixation device, etc.), etc. A determination may then be made in operation 302 as to whether to calibrate position sensing. If in operation 302 it is determined to perform position sensing, then in operation 304 at least the position sensor may be calibrated. Operation 306 may follow a determination in operation 302 not to perform positioning sensing or operation 304. Operations 306 to 308 may occur if the system has the ability to receive updates including at least the therapeutic position. In operation 306 a determination may be made as to whether the therapeutic position needs to be updated (e.g., in view of a change in treatment prescribed by a medical practitioner). If in operation 306 it is determined that the therapeutic position should be updated, then in operation 308 an updated therapeutic position may be received from an external system (e.g., via wired or wireless communication).

Operation 310 may follow a determination in operation 306 not to update the therapeutic position or operation 308. In operation 310 a position of a body part on which the sensor is worn may be determined. Operation 310 may include, for example, using the sensor to sense the body part, generating an electronic signal based on the sensing and then determining a position for the body part based at least on the electronic signal. Operations 312 to 314 may occur if the system has the ability to store and then export determined positions of the body part. In operation 312 the position of the body part determined in operation 310 may be stored. A determination may then be made in operation 314 as to whether to export some or all of the stored position data for the body part. If in operation 314 it is determined that position data should be exported, then in operation 316 some or all of the stored body part position data may be transmitted to an external system. Operation 318 may follow a determination in operation 314 to not export any body part position data or operation 316. A determination may be made in operation 318 as to whether a position change is required with respect to the body part. The determination in operation 318 may be made by, for example, comparing the position of the body part determined in operation 310 to the therapeutic position. A determination in operation 318 that no change is required may be followed by a return to operation 310 to continue sensing the position of the body part. If in operation 318 it is determined that a position change is required, then in operation 320 a notification may be generated instructing the patient how to move the body part. Operation 320 may be followed by a return to operation 310 to continue sensing the position of the body part.

While FIG. 3 illustrates operations according to an embodiment, it is to be understood that not all of the operations depicted in FIG. 3 are necessary for other embodiments. Indeed, it is fully contemplated herein that in other embodiments of the present disclosure, the operations depicted in FIG. 3, and/or other operations described herein, may be combined in a manner not specifically shown in any of the drawings, but still fully consistent with the present disclosure. Thus, claims directed to features and/or operations that are not exactly shown in one drawing are deemed within the scope and content of the present disclosure.

As used in this application and in the claims, a list of items joined by the term “and/or” can mean any combination of the listed items. For example, the phrase “A, B and/or C” can mean A; B; C; A and B; A and C; B and C; or A, B and C. As used in this application and in the claims, a list of items joined by the term “at least one of” can mean any combination of the listed terms. For example, the phrases “at least one of A, B or C” can mean A; B; C; A and B; A and C; B and C; or A, B and C.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage mediums. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums (e.g., non-transitory storage mediums) having stored thereon, individually or in combination, instructions that when executed by one or more processors perform the methods. Here, the processor may include, for example, a server CPU, a mobile device CPU, and/or other programmable circuitry. Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), embedded multimedia cards (eMMCs), secure digital input/output (SDIO) cards, magnetic or optical cards, or any type of media suitable for storing electronic instructions. Other embodiments may be implemented as software modules executed by a programmable control device.

Thus, the present disclosure pertains to a system to facilitate therapeutic positioning for a body part. An example system may be wearable by a user (e.g., a patient) and may comprise at least a sensor and presentation circuitry. The sensor may sense a position corresponding to a body part of the patient and generate an electronic signal based on the position. The presentation circuitry may then present a notification (e.g., display visible indicia and/or generate sound) to instruct the patient how to move the body part into a therapeutic position. For example, control circuitry in the system may receive the electronic signal from the sensor, compare the determined position to the therapeutic position, generate the notification and provide the notification to the presentation circuitry.

The following examples pertain to further embodiments. The following examples of the present disclosure may comprise subject material such as a device, a method, at least one machine-readable medium for storing instructions that when executed cause a machine to perform acts based on the method, means for performing acts based on the method and/or a system to facilitate therapeutic positioning for a body part, as provided below.

According to example 1 there is provided a system for therapeutic positioning of a body part. The system may comprise a sensor wearable by a user, wherein the sensor is to sense a position of a user body part on which the sensor is worn and generate an electronic signal indicative of the position and presentation circuitry wearable by the user, wherein the presentation circuitry is to present at least one notification instructing how the body part should be moved based on the electronic signal to relocate the body part from the position to a therapeutic position.

Example 2 may include the elements of example 1, wherein at least a portion of the presentation circuitry is located in a first presentation device positioned in front of an eye of the user and the notification causes the first presentation device to present visible indicia instructing how to move the body part.

Example 3 may include the elements of example 2, wherein the first presentation device comprises at least a display to present the visible indicia.

Example 4 may include the elements of example 3, wherein the display comprises at least an array of light emitting diodes (LEDs) that, when illuminated, visually instruct the user how to move the body part.

Example 5 may include the elements of any of examples 2 to 4, wherein the first presentation device comprises the sensor.

Example 6 may include the elements of any of examples 2 to 5, wherein at least a portion of the presentation circuitry is located in a second presentation device to at least generate sound and the notification causes the second presentation device to generate audible indications instructing how to move the body part.

Example 7 may include the elements of example 6, and may further comprise control circuitry to at least receive the electronic signal, determine the position of the body part based on the electronic signal, generate the notification based on the position and provide the notification to the presentation circuitry.

Example 8 may include the elements of example 7, and may further comprise communication circuitry coupled to at least the control circuitry to at least one of receive data regarding at least the therapeutic position or transmit data regarding the determined position of the body part.

Example 9 may include the elements of example 8, wherein at least the control circuitry and communication circuitry are located in a data processing device wearable by the user, the control circuitry interacting with at least one of the first or second presentation circuitry via the communication circuitry.

Example 10 may include the elements of any of examples 7 to 9, and may further comprise user interface circuitry coupled to at least the control circuitry, wherein the user interface circuitry includes at least the sensor.

Example 11 may include the elements of example 10, wherein the user interface circuitry comprises at least a speaker to generate the audible indications instructing how to move the body part.

Example 12 may include the elements of any of examples 10 to 11, wherein the user interface circuitry comprises at least one control button.

Example 13 may include the elements of any of examples 7 to 12, wherein at least the sensor, the presentation circuitry and control circuitry are all coupled to an apparatus worn by the user.

Example 14 may include the elements of example 13, wherein the apparatus is a headband and the body part is the head of the user.

Example 15 may include the elements of any of examples 13 to 14, wherein the apparatus is eyeglasses, the body part is the head of the user, the first presentation device is coupled to an eyeglass lens in the eyeglasses and the control circuitry and second presentation device are coupled to a frame of the eyeglasses.

Example 16 may include the elements of example 15, wherein the apparatus is to treat the user for a detached retina.

Example 17 may include the elements of any of examples 13 to 16, wherein the apparatus includes at least one of a brace, cast or external bone fixation device and the body part is at least one limb of the user.

Example 18 may include the elements of any of examples 1 to 17, wherein the presentation circuitry is further to generate a tactile notification to alert the user that the body part is not in the therapeutic position.

According to example 19 there is provided a method for therapeutic positioning of a body part. The method may comprise activating sensing for a position of a user body part, sensing the position, generating an electronic signal corresponding to the position, comparing the position to a therapeutic position and presenting at least one notification to relocate the body part from the position to the therapeutic position based at least on the comparison.

Example 20 may include the elements of example 19, and may further comprise determining if calibration of at least a sensor for sensing the position is required and performing calibration of at least the sensor based on the determination.

Example 21 may include the elements of any of examples 19 to 20, and may further comprise determining whether to update the therapeutic position and receiving data regarding the therapeutic position from an external system based on the determination.

Example 22 may include the elements of any of examples 19 to 21, and may further comprise receiving the electronic signal, determining the position of the body part based on the electronic signal and generating the notification based on the position.

Example 23 may include the elements of any of examples 19 to 22, and may further comprise recording data corresponding to the position, determining if the data should be exported to an external system and transmitting the data to the external system based on the determination.

Example 24 may include the elements of any of examples 19 to 23, wherein presenting at least one notification comprises displaying a visible notification to the user instructing how to move the body part.

Example 25 may include the elements of any of examples 19 to 24, wherein presenting at least one notification comprises generating an audible notification to the user instructing how to move the body part.

Example 26 may include the elements of any of examples 19 to 25, wherein presenting at least one notification comprises at least one of displaying a visible notification to the user instructing how to move the body part or generating an audible notification to the user instructing how to move the body part.

According to example 27 there is provided a system for therapeutic positioning of a body part including at least one device, the system being arranged to perform the method of any of the above examples 19 to 26.

According to example 28 there is provided a chipset arranged to perform the method of any of the above examples 19 to 26.

According to example 29 there is provided at least one machine readable medium comprising a plurality of instructions that, in response to be being executed on a computing device, cause the computing device to carry out the method according to any of the above examples 19 to 26.

According to example 30 there is provided at least one device configured for therapeutic positioning of a body part, the at least one device being arranged to perform the method of any of the above examples 19 to 26.

According to example 31 there is provided a system for therapeutic positioning of a body part. The system may comprise means for activating sensing for a position of a user body part, means for sensing the position, means for generating an electronic signal corresponding to the position, means for comparing the position to a therapeutic position and means for presenting at least one notification to relocate the body part from the position to the therapeutic position based at least on the comparison.

Example 32 may include the elements of example 31, and may further comprise means for determining if calibration of at least a sensor for sensing the position is required and means for performing calibration of at least the sensor based on the determination.

Example 33 may include the elements of any of examples 31 to 32, and may further comprise means for determining whether to update the therapeutic position and means for receiving data regarding the therapeutic position from an external system based on the determination.

Example 34 may include the elements of any of examples 31 to 33, and may further comprise means for receiving the electronic signal, means for determining the position of the body part based on the electronic signal and means for generating the notification based on the position.

Example 35 may include the elements of any of examples 31 to 34, and may further comprise means for recording data corresponding to the position, means for determining if the data should be exported to an external system and means for transmitting the data to the external system based on the determination.

Example 36 may include the elements of any of examples 31 to 35, wherein the means for presenting at least one notification comprise means for displaying a visible notification to the user instructing how to move the body part.

Example 37 may include the elements of any of examples 31 to 36, wherein the means for presenting at least one notification comprise means for generating an audible notification to the user instructing how to move the body part.

Example 38 may include the elements of any of examples 31 to 37, wherein the means for presenting at least one notification comprise means for at least one of displaying a visible notification to the user instructing how to move the body part or generating an audible notification to the user instructing how to move the body part.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

What is claimed:

1. A system for therapeutic positioning of a body part, comprising:
a sensor wearable by a user, wherein the sensor is to sense a position of a user body part on which the sensor is worn and generate an electronic signal indicative of the position; and
presentation circuitry wearable by the user, wherein the presentation circuitry is to present at least one notification instructing how the body part should be moved based on the electronic signal to relocate the body part from the position to a therapeutic position.

2. The system of claim 1, wherein at least a portion of the presentation circuitry is located in a first presentation device positioned in front of an eye of the user and the notification causes the first presentation device to present visible indicia instructing how to move the body part.

3. The system of claim 2, wherein at least a portion of the presentation circuitry is located in a second presentation device to at least generate sound and the notification causes the second presentation device to generate audible indications instructing how to move the body part.

4. The system of claim 3, further comprising control circuitry to at least receive the electronic signal, determine the position of the body part based on the electronic signal, generate the notification based on the position and provide the notification to the presentation circuitry.

5. The system of claim 4, further comprising communication circuitry coupled to at least the control circuitry to at least one of receive data regarding at least the therapeutic position or transmit data regarding the determined position of the body part.

6. The system of claim 5, wherein at least the control circuitry and communication circuitry are located in a data processing device wearable by the user, the control circuitry interacting with at least one of the first or second presentation circuitry via the communication circuitry.

7. The system of claim 4, further comprising user interface circuitry coupled to at least the control circuitry, wherein the user interface circuitry includes at least the sensor.

8. The system of claim 4, wherein at least the sensor, the presentation circuitry and control circuitry are all coupled to an apparatus worn by the user.

9. The system of claim 8, wherein the apparatus is a headband and the body part is the head of the user.

10. The system of claim 8, wherein the apparatus is eyeglasses, the body part is the head of the user, the first presentation device is coupled to an eyeglass lens in the eyeglasses and the control circuitry and second presentation device are coupled to a frame of the eyeglasses.

11. A method for therapeutic positioning of a body part, comprising:
activating sensing for a position of a user body part;
sensing the position;
generating an electronic signal corresponding to the position;
comparing the position to a therapeutic position; and
presenting at least one notification to relocate the body part from the position to the therapeutic position based at least on the comparison.

12. The method of claim 11, further comprising:
determining if calibration of at least a sensor for sensing the position is required; and
performing calibration of at least the sensor based on the determination.

13. The method of claim 11, further comprising:
determining whether to update the therapeutic position; and
receiving data regarding the therapeutic position from an external system based on the determination.

14. The method of claim 11, further comprising:
receiving the electronic signal;
determining the position of the body part based on the electronic signal; and
generating the notification based on the position.

15. The method of claim 11, further comprising:
recording data corresponding to the position;
determining if the data should be exported to an external system; and
transmitting the data to the external system based on the determination.

16. The method of claim 11, wherein presenting at least one notification comprises displaying a visible notification to the user instructing how to move the body part.

17. The method of claim 11, wherein presenting at least one notification comprises generating an audible notification to the user instructing how to move the body part.

18. At least one machine-readable storage medium having stored thereon, individually or in combination, instructions for therapeutic positioning of a body part that, when executed by one or more processors, cause the one or more processors to:
activate sensing for a position of a user body part;
sense the position;
generate an electronic signal corresponding to the position;
compare the position to a therapeutic position; and
present at least one notification to relocate the body part from the position to the therapeutic position based at least on the comparison.

19. The storage medium of claim 18, further comprising instructions that, when executed by one or more processors, cause the one or more processors to:
determine if calibration of at least a sensor for sensing the position is required; and
perform calibration of at least the sensor based on the determination.

20. The storage medium of claim 18, further comprising instructions that, when executed by one or more processors, cause the one or more processors to:
determine whether to update the therapeutic position; and
receive data regarding the therapeutic position from an external system based on the determination.

21. The storage medium of claim 18, further comprising instructions that, when executed by one or more processors, cause the one or more processors to:
receive the electronic signal;
determine the position of the body part based on the electronic signal; and
generate the notification based on the position.

22. The storage medium of claim 18, further comprising instructions that, when executed by one or more processors, cause the one or more processors to:
record data corresponding to the position;
determine if the data should be exported to an external system; and
transmit the data to the external system based on the determination.

23. The storage medium of claim 18, wherein the instructions to present at least one notification comprise instructions to display a visible notification to the user instructing how to move the body part.

24. The storage medium of claim 18, wherein the instructions to present at least one notification comprise instructions to generate an audible notification to the user instructing how to move the body part.
